(19) United States
(12) Patent Application Publication
(10) Pub. No.: US 2023/0346741 A1
(43) Pub. Date: Nov. 2, 2023

(54) ERIODICTYOL COMPOSITIONS AND METHODS

(71) Applicant: CAS PROPERTIES, LLC, Newport Beach, CA (US)

(72) Inventors: Theoharis C. THEOHARIDES, Newport Beach, CA (US); James ROUFS, Newport Beach, CA (US); Paul HWANG, Newport Beach, CA (US)

(21) Appl. No.: 18/142,751

(22) Filed: May 3, 2023

Related U.S. Application Data
(60) Provisional application No. 63/316,685, filed on Mar. 4, 2022.

(51) Int. Cl. A61K 31/353; A61K 47/40; A61K 45/06; A61P 31/14

(52) U.S. Cl. CPC A61K 31/353; A61K 47/40; A61K 45/06; A61P 31/14

(57) ABSTRACT

Compositions and methods for the treatment or prevention of neurovascular inflammation and damage in a subject. The composition includes a pharmaceutically acceptable amount of eriodictyol and/or any pharmaceutically acceptable form, including salt, cocrystal, analog, or physically and/or chemically modified forms thereof.

### CROSS-REFERENCE TO RELATED APPLICATIONS

This application claims priority to, and the benefit of, U.S. Provisional Application No. 63/316,685, filed Mar. 4, 2022, titled “Eriodictyol Compositions and Methods,” the disclosure of which is incorporated herein by reference in its entirety.

### FIELD

The present disclosure is broadly concerned with compositions and methods for the treatment of neurovascular inflammation and damage in a subject, as well as conditions, diseases, and disorders arising from or related to neurovascular inflammation and damage. In particular, the present disclosure is related to compositions that include a pharmaceutically acceptable amount of eriodictyol and/or any pharmaceutically acceptable form, including salt, cocrystal, analog, or physically modified forms thereof, as well as combination treatments involving eriodictyol.

### BACKGROUND

Most chronic diseases involve disruption of protective tissue-blood barriers. Notable, but not exhaustive examples are the disruption of the skin-blood barrier leading to atopic dermatitis (eczema) and psoriasis, disruption of the gut-blood barrier leading to irritable bowel syndrome (IBS) and gluten enteropathy (celiac sprue), food allergies/intolerance, disruption of the bladder-blood barrier leading to interstitial cystitis/bladder pain syndrome (IC/BPS), disruption of the lung-blood barrier leading to asthma, and disruption of the blood-brain barrier (BBB) leading to allergies, Alzheimer's disease, amyotrophic lateral sclerosis (ALS), angiitis, asthma, atopic dermatitis, autism spectrum disorder (ASD), Long-COVID syndrome, multiple sclerosis (MS) interstitial cystitis, mast cell activation syndrome, mastocytosis, migraine headaches, myalgic encephalomyelitis/chronic fatigue syndrome (ME/CFS), perennial rhinitis, and traumatic brain injury (TBI).

In all these conditions, diseases, and disorders disruption of the protective barrier involves neurovascular inflammation and damage and increased vascular permeability leading to extravasation of circulating white blood cells, associated neurovascular inflammatory responses, disruption of neuronal connectivity, and sensitization of sensory nerves contributing to chronic neuropathic pain. These pathogenic processes involve interaction among different cell types, including but not limited to endothelial cells, lymphocytes, mast cells, microglial cells, platelets, and nerves.

Regulating the integrity of protective barriers and subsequent immune responses is critical in the prevention or treatment of the aforementioned conditions, diseases, and disorders. Available medications aim at minimizing the symptoms stemming from, rather than addressing the cause of such barrier disruption and associated neurovascular inflammation and damage. Therefore, compositions and methods of treatment for mitigation of neurovascular damage and improving patient outcomes are desirable.

### BRIEF DESCRIPTION OF THE DRAWINGS

The accompanying drawings, which are included to provide a further understanding of the embodiments of the present disclosure, are incorporated in and constitute a part of this specification, illustrate embodiments of the present disclosure, and together with the detailed description, serve to explain principles of the embodiments discussed herein. No attempt is made to show structural details of this disclosure in more detail than may be necessary for a fundamental understanding of the embodiments discussed herein and the various ways in which they may be practiced. According to common practice, the various features of the drawings discussed below are not necessarily drawn to scale. Dimensions of various features and elements in the drawings may be expanded or reduced to more clearly illustrate embodiments of the disclosure.

FIG. 1 is a graphical representation of plaque reduction data obtained by treating cells infected with Chikungunya virus with eriodictyol or nobiletin (hexamethoxyluteolin), according to an exemplary embodiment of the present disclosure.

FIG. 2 is a graphical representation of IL-1β release by microglia pretreated with either eriodictyol or nobiletin (hexamethoxyluteolin), according to an exemplary embodiment of the present disclosure.

### DETAILED DESCRIPTION

The present disclosure provides compositions and methods for the treatment of neurovascular inflammation and damage in a subject, as well as conditions, diseases and disorders arising from or related to neurovascular inflammation anddamage, or the prevention thereof. The presently disclosed compositions include eriodictyol. Eriodictyol is a flavonone compound found in several plants, including yerba santa (Eriodictyon californicum). In at least some instances, eriodictyol, also sometimes referred to as eriodictiol, is a compound having the following chemical structure shown in Structure I below and characterized as (2S)-3′,4′,5,7-Tetrahydroxyflavan-4-one or (2S)-2-(3,4-Dihydroxyphenyl)-5,7-dihydroxy-2,3-dihydro-4H-1-benzopyran-4-one:

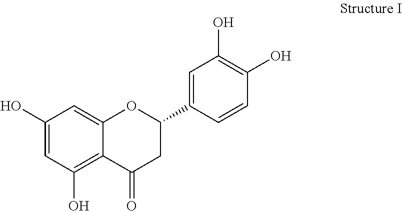 [SMILES: O=C1C[C@@H](c2ccc(O)c(O)c2)Oc2cc(O)cc(O)c21]

Eriodictyol may be used in any number of degrees of purity, including purified active pharmaceutical ingredient (API) grade. Eriodictyol may be sourced from a botanical biomass origin, including for example, as a standardized extract of Eriodictyon californicum or Citrus limon.

According to at least one aspect of the present disclosure, a composition for the treatment or prevention of neurovascular inflammation and damage in a subject is provided. The composition may include a pharmaceutically acceptable amount of eriodictyol and/or any pharmaceutically acceptable salt, cocrystal, analog, or physically modified forms thereof. Alternatively, the composition may include a pharmaceutically acceptable amount of a compound according to Structure I, and any pharmaceutically acceptable salt, cocrystal, analog, or physically modified forms thereof. The composition may also include one or more pharmaceutically acceptable excipients. In some instances, the one or more excipients may include at least one modified starch, including but not limited to cyclodextrin, as well as a lipid, such as phospholipid phosphatidylcholine.

The eriodictyol may be in any form including as a standardized extract of Eriodictyon californicum or Citrus limon. In some instances, the eriodictyol is in a microcrystalline form. In some instances, the composition may be formulated to include exosomes comprising the eriodictyol, wherein the exosomes are engineered for targeted drug delivery. In other instances, the composition may be formulated to include liposomes comprising the eriodictyol, wherein the liposomes are engineered for targeted eriodictyol delivery. Also, in other instances, the eriodictyol may be complexed, reacted, conjoined, conjugated, and/or chelated with additional compounds including, but not limited to, amino acids and derivatives thereof, and/or minerals, such as, but not limited to, zinc, calcium, magnesium, sodium, iron, manganese, and/or potassium.

In some instances, the presently disclosed compositions may include one or more exosomal carriers, the one or more exosomal carriers comprising eriodictyol and operable to provide targeted eriodictyol delivery. In other instances, the composition may include one or more liposomal carriers, the one or more liposomal carriers comprising eriodictyol and operable to provide targeted eriodictyol delivery. The presently disclosed compositions may also be formulated as a modified starch cavity-based system.

According to at least some aspects of the present disclosure, the presently disclosed compositions may include one or more additional active pharmaceutical ingredients. In some instances, the composition may include an active pharmaceutical ingredient selected from the group consisting of apigenin, fisetin, luteolin and quercetin or their methylated metabolites, tetramethoxyluteolin, hexamethoxyluteolin (nobiletin) and any combination thereof. The compositions may also include an active pharmaceutical ingredient selected from the group consisting of alpha-lipoic acid, berberine sulfate, berberine hydrochloride, L-threonine, prostaglandin precursors (e.g., arachidonic acid, di-homo-γ-linolenic acid, and eicosapentaenoic acid, and others), vitamin E, zinc, and any combination thereof. In some cases, the compositions may further include an active pharmaceutical ingredient selected from the group consisting of an anti-allergic drug, an anti-inflammatory drug, an anti-viral drug, a cytoprotective drug, and any combination thereof. The composition may also include an active pharmaceutical ingredient selected from the group consisting of benralizumab, camostat mesylase or its analogues, chloroquine or its analogues, e.g. antiallergic and antihistaminc including but not limited to azelastine, cromolyn sodium, famotidine, ketotifen, rupatadine, biologics such as dupilumab, mepolizumab, omalizumab, flamipiriravil or its analogues, antivirals such as interferon-gamma, ivermectin, ligelizumab, oseltamivir or its analogues, pemirolast or its analogues, remdesivir or its analogues, reslizumab, rifampicin or its analogues, and serine protease inhibitors including but not limited to lavgevrio and paxlovid, and any combination thereof. Additionally, the presently disclosed compositions may include an active pharmaceutical ingredient selected from the group consisting of additional zinc ionophore(s), additional flavonoids, including but not limited to methoxylated forms and derivatives thereof, and any combination thereof. Further, the compositions may include an active pharmaceutical ingredient selected from the group consisting of an anti-allergic drug, an anti-inflammatory drug, an anti-viral drug, a cytoprotective drug, and any combination thereof.

In at least some instances, the presently disclosed compositions that include eriodictyol and at least one additional active pharmaceutical ingredient exhibit a measurable synergistic effect in improved patient outcomes as compared to the eriodictyol or active pharmaceutical ingredient being administered independently.

According to an aspect of the present disclosure a method of treating or preventing neurovascular inflammation and damage in a subject is provided. The method may include administering to the subject in need thereof a pharmaceutically effective amount of one of the presently disclosed eriodictyol compositions. The neurovascular inflammation and damage in a subject may be due to a physiological response to a trigger selected from the group consisting of an allergic trigger, an environmental trigger, an inflammatory trigger, a pathogenic trigger, a stress trigger, and any combination thereof. In some instances, the neurovascular inflammation and damage in a subject may result in multiorgan failure. In some cases, the neurovascular inflammation and damage in a subject may be localized to an organ selected from the group consisting of the brain, the gut, the heart, the lungs, the skin, and any combination thereof.

In some instances, the subject treated according to the presently disclosed methods and compositions may have been diagnosed with a disorder of the brain selected from the group consisting of Alzheimer's disease, amyotrophic lateral sclerosis, angiitis, mast cell activation syndrome, migraine headaches, myalgic encephalomyelitis/chronic fatigue syndrome, Long-COVID syndrome, traumatic brain injury, and any combination thereof.

The presently disclosed methods may include administering to the subject the eriodictyol composition via an administration route selected from the group consisting of intranasally, by inhalation, oral, sublingual, transdermal, nasogastric or gastric tube routes, and any combination thereof In at least some instances, from about 1 mg to about 5000 mg of eriodictyol may be administered to the subject per day. In some cases, administering to the subject may include administering the composition such that from about 0.0125 mg to about 62.5 mg of eriodictyol per kg bodyweight is administered to the subject per day.

According to at least one aspect of the present disclosure, a method of treating or preventing a condition, disease or disorder in a subject is provided. The method may include administering to the subject in need thereof a pharmaceutically effective amount of one of the presently disclosed eriodictyol compositions. The disease or disorder may be selected from the group consisting of allergies, Alzheimer's disease, amyotrophic lateral sclerosis (ALS), angiitis, asthma, atopic dermatitis, autism spectrum disorder (ASD), Long-COVID syndrome, multiple sclerosis (MS), interstitial cystitis, mast cell activation syndrome, mastocytosis, migraine headaches, myalgic encephalomyelitis/chronic fatigue syndrome (ME/CFS), perennial rhinitis, traumatic brain injury (TBI), and any combination thereof.

According to at least one aspect of the present disclosure, a method of treating or preventing a COVID-19 associated sequelae in a subject is provided. The method may include administering to the subject in need thereof a pharmaceutically effective amount of one of the presently disclosed eriodictyol compositions. The COVID-19 associated sequelae may be selected from the group consisting of lung edema, fibrosis and inflammation, brain perivascular inflammation, heart inflammation including myocarditis, pericarditis, Long-COVID syndrome, and any combination thereof.

According to at least one aspect of the present disclosure, a method of treating or preventing COVID vaccine-associated neuroinflammatory symptoms and/or cardioinflammatory symptoms in a subject is provided. The method may include administering to the subject in need thereof a pharmaceutically effective amount of one of the presently disclosed eriodictyol compositions. The COVID vaccine-associated neuroinflammatory symptoms and/or cardioinflammatory symptoms may be selected from the group consisting of microvascular changes, platelet aggregation, myocarditis, pericarditis, thrombosis, neuroinflammatory damage, strokes, and any combination thereof.

According to at least one aspect of the present disclosure, a combination therapy for the treatment or prevention of endothelial damage in a subject is provided. The combination therapy may include administering to the subject in need thereof a composition comprising: a pharmaceutically acceptable amount of eriodictyol and/or any pharmaceutically acceptable form, including salt, cocrystal, analog, or physically modified forms thereof; and an active pharmaceutical ingredient selected from the group consisting of apigenin, fisetin, luteolin and quercetin or their methylated metabolites, tetramethoxyluteolin, hexamethoxyluteolin (nobiletin), alpha-lipoic acid, berberine sulfate, berberine hydrochloride, L-threonine, prostaglandin precursors (e.g., arachidonic acid, di-homo-γ-linolenic acid, and eicosapentaenoic acid, and others), vitamin E, zinc, and any combination thereof.

In at least some instances, the combination therapy exhibits a measurable synergistic effect in improved patient outcomes as compared to the administration of eriodictyol or the active pharmaceutical ingredient administered alone. In some instances, the subject has been diagnosed with a disease of the brain selected from the group consisting of Alzheimer's disease, amyotrophic lateral sclerosis (ALS), angiitis, autism spectrum disorder (ASD), Long- COVID syndrome, mast cell activation syndrome, migraine headaches, myalgic encephalomyelitis/chronic fatigue syndrome (ME/CFS), traumatic brain injury, and any combination thereof. In other instances, the subject has been diagnosed with a COVID-19 associated sequelae selected from the group consisting of lung edema, fibrosis and inflammation, brain perivascular inflammation, heart inflammation including, myocarditis, pericarditis, Long-COVID syndrome, and any combination thereof. In still other cases, the subject has been diagnosed with a disease, or has a disorder, is selected from the group consisting of allergies, Alzheimer's disease, amyotrophic lateral sclerosis (ALS), angiitis, asthma, atopic dermatitis, autism spectrum disorder (ASD), encephalitis, Long-COVID syndrome, multiple sclerosis (MS), interstitial cystitis, mast cell activation syndrome, mastocytosis, migraine headaches, myalgic encephalomyelitis/chronic fatigue syndrome (ME/CFS), rhinitis, traumatic brain injury (TBI), and any combination thereof.

### EXAMPLES

The examples provided below illustrate selected aspects of the presently disclosed compositions and methods.

### Example 1-Treatment of RNA Viral Infection Plaques with Eriodictyol

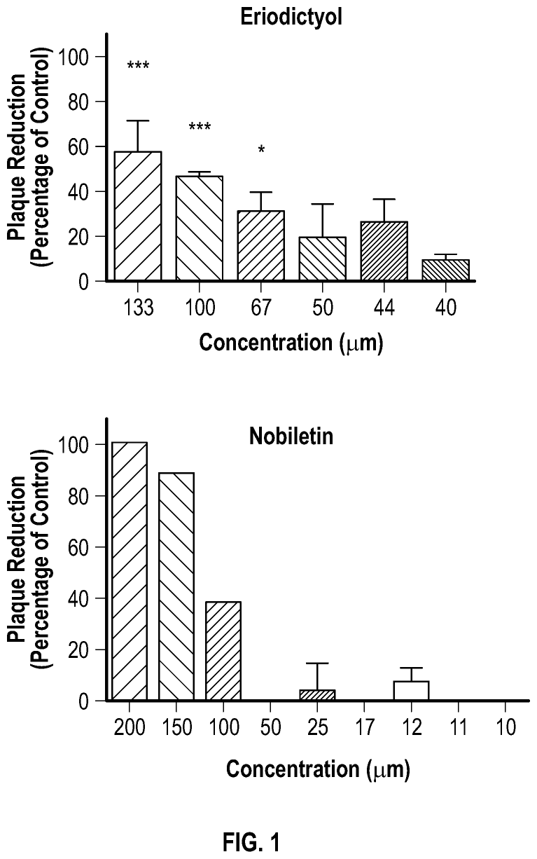

Human microglia cells infected with Chikungunya virus were treated with eriodictyol or nobiletin (hexamethoxyluteolin) in order to demonstrate the effect of eriodictyol treatment on RNA viral infection plaques. Cultures of human microglia cells were used because microglia are the brain's innate immune system cells and their activation is implicated in all neuroinflammatory and neurogenerative diseases. Chikungunya was chosen as a suitable RNA virus surrogate for coronavirus. The data obtained from treatment of viral plaques with eriodictyol or nobiletin is shown in FIG. 1. As shown in FIG. 1, treatment of the cells with eriodictyol resulted in a significant plaque reduction in a dose-dependent manner. Further, eriodictyol was found to be a slightly more potent inhibitor than nobiletin at 100 micromolar concentration.

### Example 2-Pretreatment of Human SV40 Microglia Cultures with Eriodictyol

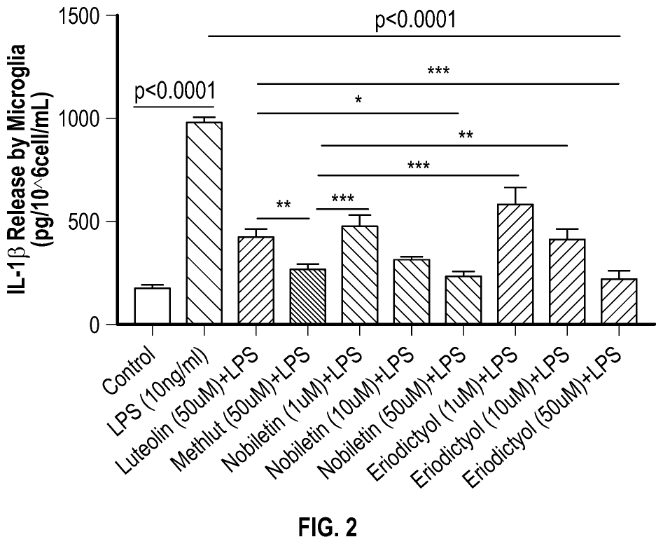

Cultured human SV40 microglia cells were pretreated with eriodictyol (Erio) or nobiletin (Nobi) for two hours to determine the degree to which eriodictyol and nobiletin inhibits the secretion of the proinflammatory cytokine interleukin-1β(IL-1β) followed by stimulation by bacteria lipopolysaccharide (LPS). The data obtained from the experiments is shown in FIG. 2. As shown in FIG. 2, eriodictyol, as well as nobiletin, was found to be more potent inhibitors of IL-1β secretion as compared to luteolin (Lut) and methoxyluteolin (Methlut) at 50 micromolar concentration.

### Example 3-Treatment of Long-Covid Syndrome with Eriodictyol

A 33 year-old Caucasian female subject having a diagnosis of long-COVID syndrome was administered 60 milligrams (mg) eriodictyol (30 mg/vegetarian capsule; 2 capsules daily) per day for three months. The subject reported an over 80% reduction in long-covid symptoms that included a 80% reduction in fatigue, brain fog, difficulty concentrating, difficulty remembering, difficulty finding the right words, and difficulty multi-tasking.

Statements of the Disclosure:
Statement 1: A composition for the treatment or prevention of neurovascular inflammation and damage in a subject, the composition comprising: a pharmaceutically acceptable amount of eriodictyol and/or any pharmaceutically acceptable form, including salt, cocrystal, analog, or physically modified forms thereof.
Statement 2: A composition for the treatment or prevention of endothelial damage in a subject, the composition comprising: a pharmaceutically acceptable amount of a compound according to Structure I, and any pharmaceutically acceptable form, including salt, cocrystal, analog, or physically modified forms thereof:

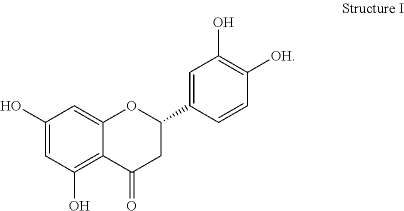 [SMILES: O=C1C[C@@H](c2ccc(O)c(O)c2)Oc2cc(O)cc(O)c21]

Statement 3: The composition according to Statement 1 or Statement 2, further comprising one or more pharmaceutically acceptable excipients.
Statement 4: The composition according to Statements 3, wherein the one or more excipients comprises at least one modified starch.
Statement 5: The composition according to Statement 3 and Statement 4, wherein the at least one modified starch is cyclodextrin.
Statement 6: The composition according to any one of Statements 1-5, wherein the eriodictyol is a standardized extract of Eriodictyon californicum or Citrus limon.
Statement 7: The composition according to any one of Statements 1-6, wherein the eriodictyol is in a microcrystalline form.
Statement 8: The composition according to any one of Statements 1-7, formulated to include exosomes comprising the eriodictyol, wherein the exosomes are engineered for targeted drug delivery.
Statement 9: The composition according to any one of Statements 1-7, formulated to include liposomes comprising the eriodictyol, wherein the liposomes are engineered for targeted eriodictyol delivery.
Statement 10: The composition according to any one of Statements 1-7, comprising one or more exosomal carriers, the one or more exosomal carriers comprising eriodictyol and operable to provide targeted eriodictyol delivery.
Statement 11: The composition according to any one of Statements 1-7, comprising one or more liposomal carriers, the one or more liposomal carriers comprising eriodictyol and operable to provide targeted eriodictyol delivery.
Statement 12: The composition according to any one of Statements 1-11, formulated as a modified starch cavity-based system.
Statement 13: The composition according to any one of Statements 1-12, further comprising an active pharmaceutical ingredient selected from the group consisting of apigenin, fisetin, luteolin and quercetin or their methylated metabolites, tetramethoxyluteolin, hexamethoxyluteolin (nobiletin) and any combination thereof.
Statement 14: The composition according to any one of Statements 1-13, further comprising an active pharmaceutical ingredient selected from the group consisting of alpha-lipoic acid, berberine sulfate, berberine hydrochloride, L-threonine, prostaglandin precursors (e.g., arachidonic acid, di-homo-γ-linolenic acid, and eicosapentaenoic acid, and others), vitamin E, zinc, and any combination thereof.
Statement 15: The composition according to any one of Statements 1-14, further comprising an active pharmaceutical ingredient selected from the group consisting of an anti-allergic drug, an anti-inflammatory drug, an anti-viral drug, a cytoprotective drug, and any combination thereof.
Statement 16: The composition according to any one of Statements 1-15, further comprising an active pharmaceutical ingredient selected from the group consisting of benralizumab, camostat mesylase or its analogues, chloroquine or its analogues, antiallergic and antihistaminics including but not limited to azelastine, cromolyn sodium, famotidine, ketotifen, rupatadine; antioxidants including but not limited to curcumin, oleorupein, hydroxytyrosol, resveratrol, sulforaphane; biologics such as dupilumab, mepolizumab, omalizumab, flamipiriravil or its analogues; antivirals such as interferon-gamma, ivermectin, ligelizumab, oseltamivir or its analogues, pemirolast or its analogues, remdesivir or its analogues, reslizumab, rifampicin or its analogues, and serine protease inhibitors including but not limited to lavgevrio and paxlovid, and any combination thereof.
Statement 17: The composition according to any one of Statements 1-16, further comprising an active pharmaceutical ingredient selected from the group consisting of additional zinc ionophore(s), additional flavonoids, including but not limited to methoxylated forms and derivatives thereof, and any combination thereof.
Statement 18: The composition according to any one of Statements 1-14, further comprising an active pharmaceutical ingredient selected from the group consisting of an anti-allergic drug, an anti-inflammatory drug, an anti-viral drug, a cytoprotective drug, and any combination thereof.
Statement 19: The composition according to any one of Statements 13-18, wherein the composition comprising eriodictyol and at least one additional active pharmaceutical ingredient exhibits a measurable synergistic effect in improved patient outcomes.
Statement 20: A method of treating or preventing neurovascular inflammation and damage in a subject, the method comprising: administering to the subject in need thereof a pharmaceutically effective amount of the composition according to any one of Statements 1-19.
Statement 21: A method according to Statement 20, wherein the neurovascular inflammation and damage in a subject is due to a physiological response to a trigger selected from the group consisting of an allergic trigger, an environmental trigger, an inflammatory trigger, a pathogenic trigger, a stress trigger, and any combination thereof.
Statement 22: The method according to Statement 20 or Statement 21, wherein the neurovascular inflammation and damage in a subject results in multiorgan failure.
Statement 23: The method according to any one of Statements 20-22, wherein the neurovascular inflammation and damage in a subject is localized to an organ selected from the group consisting of the brain, the gut, the heart, the lungs, the skin, and any combination thereof
Statement 24: The method according to any one of Statements 20-23, wherein the subject has been diagnosed with a disorder of the brain selected from the group consisting of Alzheimer's disease, amyotrophic lateral sclerosis (ALS), angiitis, autism spectrum disorder (ASD), encephalitis, Long-COVID syndrome, mast cell activation syndrome, migraine headaches, multiple sclerosis (MS), myalgic encephalomyelitis/chronic fatigue syndrome (ME/CFS), traumatic brain injury, and any combination thereof.
Statement 25: The method according to any one of Statements 20-24, wherein administering to the subject comprises administering via an administration route selected from the group consisting of intranasally, by inhalation, oral, sublingual, transdermal, nasogastric or gastric tube routes, and any combination thereof.
Statement 26: The method according to any one of Statements 20-25, wherein administering to the subject comprises administering the composition such that from about 1 mg to about 5000 mg of eriodictyol is administered to the subject per day.
Statement 27: The method according to any one of Statements 20-25, wherein administering to the subject comprises administering the composition such that from about 0.0125 mg to about 62.5 mg of eriodictyol per kg bodyweight is administered to the subject per day.
Statement 28: A method of treating or preventing a disease, or a disorder, in a subject, the method comprising: administering to the subject in need thereof a pharmaceutically effective amount of the composition according to any one of Statements 1-19 wherein the condition, disease or disorder is selected from the group consisting of allergies, Alzheimer's disease, amyotrophic lateral sclerosis (ALS), angiitis, asthma, atopic dermatitis, autism spectrum disorder (ASD), Long-COVID syndrome, encephalitis, multiple sclerosis (MS), interstitial cystitis, mast cell activation syndrome, mastocytosis, migraine headaches, myalgic encephalomyelitis/chronic fatigue syndrome (MS/CFS), rhinitis, traumatic brain injury (TBI), and any combination thereof.
Statement 29: The method according to any one of Statement 28, wherein administering to the subject comprises administering via an administration route selected from the group consisting of intranasally, by inhalation, oral, sublingual, transdermal, nasogastric or gastric tube routes, and any combination thereof.
Statement 30: The method according to Statement 28 or Statement 29, wherein administering to the subject comprises administering the composition such that from about 1 mg to about 5000 mg of eriodictyol is administered to the subject per day.
Statement 31: The method according to any one of Statements 28-30, wherein administering to the subject comprises administering the composition such that from about 0.0125 mg to about 62.5 mg of eriodictyol per kg bodyweight is administered to the subject per day.
Statement 32: A method of treating or preventing a COVID-19 associated sequela in a subject, the method comprising: administering to the subject in need thereof a pharmaceutically effective amount of the composition according to any one of Statements 1-19.
Statement 33: The method according to any one of Statement 32, wherein the COVID-19 associated sequelae is selected from the group consisting of lung edema, fibrosis and inflammation, brain perivascular inflammation, heart inflammation including, myocarditis, pericarditis, Long-COVID syndrome, and any combination thereof.
Statement 34: The method according to Statement 32 or Statement 33, wherein administering to the subject comprises administering via an administration route selected from the group consisting of intranasally, by inhalation, oral, sublingual, transdermal, nasogastric or gastric tube routes, and any combination thereof.
Statement 35: The method according to any one of Statements 32-34, wherein administering to the subject comprises administering the composition such that from about 1 mg to about 5000 mg of eriodictyol is administered to the subject per day.
Statement 36: The method according to any one of Statements 32-35, wherein administering to the subject comprises administering the composition such that from about 0.0125 mg to about 62.5 mg of eriodictyol per kg bodyweight is administered to the subject per day.
Statement 37: A method of treating or preventing COVID vaccine-associated neuroinflammatory symptoms and/or cardioinflammatory symptoms in a subject, the method comprising: administering to the subject in need thereof a pharmaceutically effective amount of the composition according to any one of Statements 1-19.
Statement 38: The method according to Statement 37, wherein the COVID vaccine-associated neuroinflammatory symptoms and/or cardioinflammatory symptoms is selected from the group consisting of microvascular changes, platelet aggregation, myocarditis, pericarditis, thrombosis, neuroinflammatory damage, strokes, and any combination thereof.
Statement 39: The method according to Statement 37 or Statement 38, wherein administering to the subject comprises administering via an administration route selected from the group consisting of intranasally, by inhalation, oral, sublingual, transdermal, nasogastric or gastric tube routes, and any combination thereof.
Statement 40: The method according to any one of Statements 37-39, wherein administering to the subject comprises administering the composition such that from about 1 mg to about 5000 mg of eriodictyol is administered to the subject per day.
Statement 41: The method according to any one of Statements 37-40, wherein administering to the subject comprises administering the composition such that from about 0.0125 mg to about 62.5 mg of eriodictyol per kg bodyweight is administered to the subject per day.
Statement 42: A combination therapy for the treatment or prevention of endothelial damage in a subject, the combination therapy comprising: administering to the subject in need thereof a composition comprising: a pharmaceutically acceptable amount of eriodictyol and/or any pharmaceutically acceptable form, including salt, cocrystal, analog, or physically modified forms thereof; and an active pharmaceutical ingredient selected from the group consisting of apigenin, fisetin, luteolin and quercetin or their methylated metabolites, tetram ethoxyluteol in, alpha-lipoic acid, berberine sulfate, berberine hydrochloride, L-threonine, prostaglandin precursors (e.g., arachidonic acid, di-homo-γ-linolenic acid, and eicosapentaenoic acid, and others), vitamin E, zinc, and any combination thereof.
Statement 43: The combination therapy according to Statement 42, wherein the endothelial damage in a subject is due to a physiological response to a trigger selected from the group consisting of an allergic trigger, an environmental trigger, an inflammatory trigger, a pathogenic trigger, a stress trigger, and any combination thereof.
Statement 44: The combination therapy according to Statement 41 or Statement 42, wherein the endothelial damage in a subject results in multiorgan failure.
Statement 45: The combination therapy according to any one of Statements 41-44, wherein the endothelial damage in a subject is localized to an organ selected from the group consisting of the brain, the heart, the lungs, the skin, and any combination thereof.
Statement 46: The combination therapy according to any one of Statements 41-45, wherein the subject has been diagnosed with a disease, or a disorder, of the brain selected from the group consisting of Alzheimer's disease, amyotrophic lateral sclerosis, angiitis, autism spectrum disorder (ASD), mast cell activation syndrome, migraine headaches, myalgic encephalomyelitis/chronic fatigue syndrome, Long-COVID syndrome, traumatic brain injury, and any combination thereof.
Statement 47: The combination therapy according to any one of Statements 41-46, wherein administering to the subject comprises administering via an administration route selected from the group consisting of intranasally, by inhalation, oral, sublingual, transdermal, nasogastric or gastric tube routes, and any combination thereof.
Statement 48: The combination therapy according to any one of Statements 41-47, wherein administering to the subject comprises administering the composition such that from about 1 mg to about 5000 mg of eriodictyol is administered to the subject per day.
Statement 49: The combination therapy according to any one of Statements 41-47, wherein administering to the subject comprises administering the composition such that from about 0.0125 mg to about 62.5 mg of eriodictyol per kg bodyweight is administered to the subject per day.
Statement 50: The combination therapy according to any one of Statements 41-49, wherein the combination therapy exhibits a measurable synergistic effect in improved patient outcomes as compared to the administration of eriodictyol or the active pharmaceutical ingredient administered alone.
Statement 51: The combination therapy according to any one of Statements 41-50, wherein the subject has been diagnosed with a COVID-19 associated sequelae selected from the group consisting of lung edema, fibrosis and inflammation, brain perivascular inflammation, heart inflammation including, myocarditis, pericarditis, Long-COVID syndrome, and any combination thereof.
Statement 52: The combination therapy according to any one of Statements 41-50, wherein the subject has been diagnosed with a condition, disease, or a disorder, is selected from the group consisting of allergies, Alzheimer's disease, amyotrophic lateral sclerosis (ALS), angiitis, asthma, atopic dermatitis, autism spectrum disorder (ASD), Long-COVID syndrome, multiple sclerosis (MS), interstitial cystitis, mast cell activation syndrome, mastocytosis, migraine headaches, myalgic encephalomyelitis/chronic fatigue syndrome (ME/CFS), perennial rhinitis, traumatic brain injury (TBI), and any combination thereof.

1. A composition for the treatment or prevention of neurovascular inflammation and damage in a subject, the composition comprising:
a pharmaceutically acceptable amount of eriodictyol and/or any pharmaceutically acceptable form, including salt, cocrystal, analog, or physically modified forms thereof

2. The composition according to claim 1, further comprising cyclodextrin.

3. The composition according to claim 1, comprising one or more exosomal carriers, the one or more exosomal carriers comprising eriodictyol and operable to provide targeted eriodictyol delivery.

4. The composition according to claim 1, further comprising an active pharmaceutical ingredient selected from the group consisting of apigenin, fisetin, luteolin and quercetin or their methylated metabolites, tetramethoxyluteolin, hexamethoxyluteolin (nobiletin), and any combination thereof.

5. The composition according to claim 1, further comprising an active pharmaceutical ingredient selected from the group consisting of alpha-lipoic acid, berberine sulfate, berberine hydrochloride, L-threonine, arachidonic acid, di-homo-y-linolenic acid, eicosapentaenoic acid, vitamin E, zinc, and any combination thereof.

6. The composition according to claim 1, further comprising an active pharmaceutical ingredient selected from the group consisting of benralizumab, camostat mesylase or its analogues, chloroquine or its analogues, antiallergic and antihistaminics including but not limited to azelastine, cromolyn sodium, famotidine, ketotifen, rupatadine; antioxidants including but not limited to curcumin, oleorupein, hydroxytyrosol, resveratrol, sulforaphane; biologics such as dupilumab, mepolizumab, omalizumab, flamipiriravil or its analogues; antivirals such as interferon-gamma, ivermectin, ligelizumab, oseltamivir or its analogues, pemirolast or its analogues, remdesivir or its analogues, reslizumab, rifampicin or its analogues, and serine protease inhibitors including but not limited to lavgevrio and paxlovid, and any combination thereof.

7. The composition according to claim 1, further comprising an active pharmaceutical ingredient selected from the group consisting of additional zinc ionophore(s), additional flavonoids, including but not limited to methoxylated forms and derivatives thereof, and any combination thereof.

8. The composition according to claim 1, further comprising an active pharmaceutical ingredient selected from the group consisting of an anti-allergic drug, an anti-inflammatory drug, an anti-viral drug, a cytoprotective drug, and any combination thereof.

9. The composition according to claim 1, wherein the composition comprising eriodictyol and at least one additional active pharmaceutical ingredient exhibits a measurable synergistic effect in improved patient outcomes.

10. A method of treating or preventing neurovascular inflammation and damage in a subject, the method comprising:
administering to the subject in need thereof a pharmaceutically effective amount of the composition according to claim 1.

11. The method according to claim 10, wherein the neurovascular inflammation and damage in a subject is due to a physiological response to a trigger selected from the group consisting of an allergic trigger, an environmental trigger, an inflammatory trigger, a pathogenic trigger, a stress trigger, and any combination thereof.

12. The method according to claim 10, wherein the neurovascular inflammation and damage in a subject results in multiorgan failure.

13. The method according to claim 10, wherein the neurovascular inflammation and damage in a subject is localized to an organ selected from the group consisting of the brain, the gut, the heart, the lungs, the skin, and any combination thereof.

14. The method according to claim 10, wherein the subject has been diagnosed with a disorder of the brain selected from the group consisting of Alzheimer's disease, amyotrophic lateral sclerosis (ALS), angiitis, autism spectrum disorder (ASD), encephalitis, Long-COVID syndrome, mast cell activation syndrome, migraine headaches, multiple sclerosis (MS), myalgic encephalomyelitis/chronic fatigue syndrome (ME/CFS), traumatic brain injury, and any combination thereof.

15. The method according to claim 10, wherein administering to the subject comprises administering the composition such that from about 0.0125 mg to about 62.5 mg of eriodictyol per kg bodyweight is administered to the subject per day.

16. A method of treating or preventing a disease, or a disorder, in a subject, the method comprising:
administering to the subject in need thereof a pharmaceutically effective amount of the composition according to claim 1 wherein the condition, disease or disorder is selected from the group consisting of allergies, Alzheimer's disease, amyotrophic lateral sclerosis (ALS), angiitis, asthma, atopic dermatitis, autism spectrum disorder (ASD), Long-COVID syndrome, encephalitis, multiple sclerosis (MS), interstitial cystitis, mast cell activation syndrome, mastocytosis, migraine headaches, myalgic encephalomyelitis/chronic fatigue syndrome (MS/CFS), rhinitis, traumatic brain injury (TBI), and any combination thereof.

17. The method according to claim 16, wherein the condition, disease or disorder is long-covid syndrome.

18. A method of treating or preventing a COVID-19 associated sequela in a subject, the method comprising:
administering to the subject in need thereof a pharmaceutically effective amount of the composition according to claim 1.

19. The method according to claim 18, wherein the COVID-19 associated sequelae is selected from the group consisting of lung edema, fibrosis and inflammation, brain perivascular inflammation, heart inflammation including, myocarditis, pericarditis, Long- COVID syndrome, and any combination thereof.

20. A combination therapy for the treatment or prevention of endothelial damage in a subject, the combination therapy comprising:
administering to the subject in need thereof a composition comprising:
a pharmaceutically acceptable amount of eriodictyol or any pharmaceutically acceptable form, including salt, cocrystal, analog, or physically modified forms thereof; and
an active pharmaceutical ingredient selected from the group consisting of hexamethoxyluteolin (nobiletin), apigenin, fisetin, luteolin and quercetin or their methylated metabolites, tetramethoxyluteolin, alpha-lipoic acid, berberine sulfate, berberine hydrochloride, L-threonine, prostaglandin precursors (e.g., arachidonic acid, di-homo-y-linolenic acid, and eicosapentaenoic acid, and others), vitamin E, zinc, and any combination thereof.
